(19) United States
(12) Patent Application Publication
(10) Pub. No.: US 2011/0052626 A1
(43) Pub. Date: Mar. 3, 2011

(54) LEPORIPOXVIRUS-DERIVED VACCINE VECTORS

(76) Inventors: Stéphane Bertagnoli, Lavernose-Lacasse (FR); Béatrice Pignolet, Toulouse (FR); Jacqueline Gelfi, Tournefeuille (FR); Christelle Camus-Bouclainville, Toulouse (FR)

(21) Appl. No.: 12/808,971

(22) PCT Filed: Dec. 3, 2008
(86) PCT No.: PCT/FR2008/001678

§ 371 Date: Aug. 18, 2010

(30) Foreign Application Priority Data
Dec. 18, 2007 (FR) 07/08826

(51) Int. Cl. A61K 39/275; C12N 7/01; C07H 21/04; C12N 15/63; A61P 31/20

(52) U.S. Cl. 424/199.1; 435/235.1; 536/23.72; 435/320.1

(57) ABSTRACT

The invention relates to modified leporipoxyinises expressing a chimeric envelope protein resulting from the fusion of an envelope protein of mature intracellular virions or of an envelope protein of enveloped extracellular virions, with an exogenous protein. These modified leporipoxviruses can be used in particular for obtaining vaccines.

The present invention relates to novel vaccine vectors derived from Leporipoxvirus and especially from the myxomatosis virus.

The Leporipoxvirus genus belongs to the Poxyiridae family. This genus, the standard species of which is the myxomatosis virus (MV), collates poxviruses specific to the Leporidae. Myxomatosis virus, also known as Myxoma virus, is the agent responsible for myxomatosis, a major infectious disease in European rabbits, which is endemic in Europe. Poxyiridae has an intracytoplasmic replicative cycle during which various viral forms are successively observed: IMV (intracellular mature virions), IEV (intracellular envelope virions) and EEV (extracellular envelope virions).

IMVs are formed in the cytoplasma of infected cells, and have a single membrane. They represent the vast majority of the viral progeny and are released during lysis of the host cell. A small portion of IMVs acquires an additional double envelope derived from the Golgi apparatus or from endosomes, constituting the IEV viral forms. The IEVs migrate to the cell surface and fuse with the cell membrane. They thus lose their outermost envelope and release EEVs into the extracellular medium.

Recombinant poxviruses have shown their efficacy as vaccination vectors by their capacity for producing an immune response against foreign antigens. Their exclusively cytoplasmic replicative cycle makes it possible to avoid any complications resulting from the integration of viral DNA into the genome of the infected cell. They are capable of integrating large fragments of foreign DNA (more than 25 kbp) by homologous recombination. Furthermore, they are capable of correctly expressing the proteins of interest and of inducing a good immune response. Leporipoxviruses also have the advantage of having a very narrow in vivo host spectrum, which gives them appreciable safety of use. Moreover, although non-replicative in vitro in certain cell types (bovine, ovine, feline), they nevertheless allow the expression of transgenes. They are thus of great interest for obtaining vaccine vectors, not only in Leporidae, but also in other animal species.

Conventionally, in poxviruses, transgenes are inserted into intergene regions, or into regions containing genes not essential for the viral replication. The majority of the insertion sites that have been revealed are close to the genome ends, where genes not essential to the viral cycle are located (Mackett et al., J Virol, 49, 857-864, 1984; Perkus et al., Virology, 180, 406-410, 1991).

In the case of Leporipoxviruses, vaccine vectors that express foreign antigens have also been obtained. Bertagnoli et al. (J Virol, 70, 5061-5066, 1996), and patent application FR 2736358 describe the production of vaccine vectors derived from recombinant Myxoma viruses, which make it possible to protect rabbits both against myxomatosis and rabbit viral hemorrhagic disease. To construct these vectors, a gene coding for the VP60 capsid protein of the viral hemorrhagic disease virus was inserted into the Myxoma virus genome in the region of nonessential genes, in this case either the gene coding for thymidine kinase (TK) or the genes coding for the virulence factors MGF (myxoma growth factor) and M11L.

More recently, the use of recombinant attenuated Myxoma viruses capable of producing the capsid protein of feline calicivirose virus has shown good vaccinal efficacy in cats (McCabe et al., Vaccine, 20, 2454-2462, 2002; McCabe and Spibey, Vaccine, 23, 5380-5388, 2005). In these vectors, the transgene coding for the capsid protein was inserted into genes coding for the virulence factors MGF and M11L. Similarly, patent application PCT WO 02/072 852 describes the use of recombinant Leporipoxviruses for obtaining vaccine vectors that may be used in animals other than Leporidae, especially in birds, cats, dogs, pigs, sheep, cattle and horses, and also in man. It is indicated that, in these vectors, the transgene expressing the vaccine antigen of interest may be inserted into the gene coding for thymidine kinase, or into one of the genes coding for the virulence factors M11L, SERP-1, -2 and -3 or MGF.

The majority of the vaccines based on poxvirus vectors produce cytoplasmic antigens, which are secreted or expressed at the surface of the infected cells. Recently, it has been proposed to express vaccine antigens at the surface of viral particles, for the purpose of improving the immune response. This approach has already been used in the case of vaccine vectors derived from various viruses, for example poliovirus (Mandl et al., Proc. Natl. Acad. Sci. USA, 95, 8216-8221, 1998), hepatitis B virus (Ulrich et al., Adv. Virus Res., 50, 141-182, 1998), or hepatitis A virus (Kusov et al., Antiviral Res., 73, 101-111, 2007). As regards poxviruses, the only vectors of this type described to date were obtained from recombinant vaccine viruses in which the vaccine antigens of interest are fused to the B5R envelope protein of the EEV virions (Katz and Moss, AIDS Res. Hum. Retroviruses, 13, 1497-1500, 1997; Barchichat and Katz, Virus Res., 90, 243-251, 2002; Kwak et al., Virology, 322, 337-348, 2004).

This approach was not able to be envisioned hitherto in the case of Leporipoxviruses, since they do not have B5R protein homology.

The Inventors have now identified two Leporipoxvirus envelope proteins to which it is possible to fuse an antigen of interest, without this fusion impairing the functions of these proteins in the viral morphogenesis.

These are the proteins named M071L and M022L in the myxomatosis virus (Cameron et al., Virology, 264, 298-318, 1999).

M071L (GenBank: NP_{—}051785, GI:9633707) is a 324-aa protein encoded by a late gene; it has a molecular mass of about 36.8 kDa; it is integrated into the IMV membrane, of which it constitutes the immunodominant antigenic component.

Its homolog in another Leporipoxvirus, rabbit fibroma virus (Willer et al., Virology, 264, 319-343, 1999), is known as gp071L (NP_{—}051960, GI:9633880). Its homolog in vaccinia virus is the H3L protein, which is involved in the maturation of viral particles (Da Fonseca et al., J. Virol., 74, 7518-7528, 2000). M071L has 90% identity and 94% sequence similarity with gp071L and 35% identity and 61% similarity with H3L.

M022L (GenBank NP_{—}051736, GI:9633658) is the predominant protein present on the envelope of the EEV viral forms. It is a 371-aa protein with a molecular mass of about 41.5 kDa. Its homolog in rabbit fibroma virus is known as gp022L (NP_{—}051911, GI:9633833). Its homolog in vaccinia virus is the F13L protein, which is involved in IEV membrane formation (Husain and Moss, J. Virol, 75, 7528-7542, 2001). M022L has 92% identity and 95% sequence similarity with gp022L and 55% identity and 71% similarity with F13L.

The sequence of the M071L protein of myxomatosis virus (identical to the sequence GenBank NP_{—}051785) is represented in the attached sequence listing under the number SEQ ID NO: 1; the sequence of the M022L protein of myxomatosis virus (identical to the sequence GenBank NP_{—}051736) is represented in the attached sequence listing under the number SEQ ID NO: 2.

The Inventors have found that when a sequence coding for an exogenic protein is fused to the sequence coding for M071L, so as to obtain a chimeric protein comprising said exogenic protein fused to the C-terminal end of M071L, this chimeric protein is expressed comparable to the native M071L protein. In addition, this C-terminal fusion does not interfere with the function of the M071L protein in viral morphogenesis, and in modified Myxoma viruses, expressing the chimeric protein, it is localized as the native M071L protein, on the IMV membrane. The exogenic protein is presented at the surface of the IMVs and may also induce an immune response.

The Inventors have also found, on the other hand, that when the exogenic protein is fused to the N-terminal end of M071L, it is not possible to obtain a recombinant virus that expresses the fused protein, suggesting a lethal modification of the M071L protein.

In the case of M022L, the Inventors have found that the fusion of an exogenic protein to its C-terminal end produces a nonfunctional chimeric protein, and results in the absence of formation of viral particles. On the other hand, when the exogenic protein is fused to the N-terminal end of M022L, the chimeric protein obtained conserves, in modified Myxoma viruses that express this protein, the function and localization of the native M022L protein, and allows presentation of the exogenic protein at the surface of EEVs and the induction of a humoral and cellular immune response directed against this exogenic protein.

In addition, the Inventors have observed that modified Myxoma viruses that express the chimeric proteins described above, although having a morphology and a replication capacity comparable to those of the wild-type strain from which they are derived, have a considerably attenuated pathogenic power.

One subject of the present invention is, consequently, a modified Leporipoxvirus, characterized in that it expresses a chimeric envelope protein chosen from:

a) a chimeric envelope protein comprising:
a region A formed by a protein having at least 85%, preferably at least 90% and most preferably at least 95% identity, or at least 90%, preferably at least 92% and most preferably at least 96% sequence similarity with the M071L protein defined by the sequence SEQ ID NO: 1;
a region B formed by an exogenic protein of interest, said region B being fused to the C-terminal end of said region A;

b) a chimeric envelope protein comprising:
a region A formed by a protein having at least 85%, preferably at least 90% and most preferably at least 95% identity, or at least 90%, preferably at least 92% and most preferably at least 96% sequence similarity with the M022L protein defined by the sequence SEQ ID NO: 2;
a region B formed by an exogenic protein of interest, said region B being fused to the N-terminal end of said region A, directly, or optionally via a peptide linker of a few amino acids.

Unless otherwise indicated, the percentages of identity and of similarity to which reference is made herein are calculated by means of the BLASTP program, using the default parameters, on a comparison window formed by the whole protein sequence.

The exogenic protein of interest is expressed at the surface of the IMVs in the case of Leporipoxviruses that express a chimeric protein as defined in a) above, and at the surface of EEVs in the case of Leporipoxviruses that express a chimeric protein as defined in b) above.

According to one preferred embodiment of a modified Leporipoxvirus in accordance with the invention, it expresses a chimeric protein as defined in a) above, and a chimeric protein as defined in b) above. These two chimeric proteins may contain the same protein of interest or two different proteins of interest.

The present invention also covers any modified Leporipoxvirus that expresses a chimeric protein comprising a membrane protein of Leporipoxvirus IMV, fused with an exogenic protein of interest.

The exogenic protein of interest may be any protein against which it is desired to induce a humoral and/or cellular immune response. Its size may range from a few amino acids to a few hundred amino acids.

It may especially be an antigenic protein derived from a viral, bacterial or parasitic pathogen, or an antigenic fragment of said protein, or alternatively a recombinant protein combining various antigenic fragments, derived from one and the same antigen or from different antigens.

Leporipoxviruses modified in accordance with the invention may be obtained from wild-type Leporipoxviruses, or from attenuated Leporipoxviruses. They may especially be myxomatosis virus or the rabbit fibroma virus.

As nonlimiting examples of strains of myxomatosis virus from which modified viruses in accordance with the invention may be constructed, mention will be made of: the Lausanne strain (ATCC VR-115), the Moses strain (ATCC VR-116), the strains described in patent application FR 2 736 358, and especially the strains Leon 162 (L162): CNCM 1-1595; Hongrois (HG): CNCM 1-1593; Finistere (F9): CNCM 1-1596; R 801: CNCM 1-1598; Toulouse 1 (T1): CNCM 1-1592, and also the attenuated strain SG 33 (CNCM 1-1594).

As nonlimiting examples of strains of rabbit fibroma virus from which modified viruses in accordance with the invention may be constructed, mention will be made of: the Original A strain (Boerlage strain)(ATCC VR-112), the Patuxent strain (ATCC VR-113), the Kasza strain (ATCC VR-364).

The modified Leporipoxviruses in accordance with the invention may be produced via standard techniques used for the production of recombinant poxviruses, which involve homologous recombination between the genome of a poxvirus into which it is desired to insert a sequence of interest, and a transfer vector, containing the sequence that it is desired to insert, framed by sequences of the poxvirus genome flanking the site where the insertion is to take place. The homologous recombination occurs in host cells infected by the chosen poxvirus, and transfected by the transfer vector.

A subject of the present invention is also tools for producing modified Leporipoxviruses in accordance with the invention, and especially: a polynucleotide coding for a chimeric envelope protein, as defined above, an expression cassette containing said polynucleotide, under the control of a suitable promoter, and a recombinant vector containing said expression cassette.

Preferentially, the promoter used in the expression cassettes in accordance with the invention, the promoter is that of the gene coding for the envelope protein to which is fused the exogenous protein of interest. These expression cassettes may also comprise a marker gene that facilitates the selection of the recombinant viruses. It may be, for example, a gene that confers a selective advantage to the recombinant viruses, such as a gene for resistance to an antibiotic, such as mycophenolic acid or geneticin, or a gene enabling the visualization of lysis ranges containing the recombinant virus, such as the LacZ gene, the gus gene or a gene coding for a fluorescent protein.

Host cells that may be used for the production of the modified viruses in accordance with the invention are those usually used for the production of Leporipoxvirus; they are especially normal or tumoral rabbit cells, normal or tumoral monkey cells, normal or tumoral avian cells, or tumoral human cells.

The modified Leporipoxviruses in accordance with the invention may be used for the production of vaccines, not only in Leporidae, but also in other animal species, including birds and mammals, and especially sheep, cattle, pigs, horses, dogs, cats and primates, in particular man.

Said vaccines may be formulated for parental use, for example intradermal, intramuscular or subcutaneous use, oral use or mucous use, for example intranasal. The amount of modified virus per vaccine dose is chosen so as to allow a level of expression of the exogenous protein of interest that is sufficient to induce an immune response against this protein. It may depend especially on the nature of said exogenous protein, on the species and on the age of the individual to be vaccinated, on the type of immune response (cellular or humoral) that it is desired to favor. Usually, it will be between 10³ and 10⁹ pfu (plaque-forming units) per vaccine dose.

The present invention will be understood more clearly with the aid of the rest of the description that follows, which refers to examples illustrating the construction of modified Leporipoxviruses in accordance with the invention, and to their use in vaccination.

### EXAMPLE 1

### Construction of Recombinant Myxomatosis Viruses Expressing an Exogenous Protein in Fusion with M022L or M071L

Three recombinant viruses derived from the virus of the Toulouse 1 strain were constructed. Two produced the M022L protein fused either with GFP (Green Fluorescent Protein) or with the ectodomain of the M2 protein (M2e) (GenBank: AY340089) of avian influenza A virus in its amino-terminal part (T1-N-gfp-M022L and T1-N-M2e-M022L). The third produces the M071L protein fused with M2e in its carboxy-terminal part (T1-C-M2e-M071L).

### Materials and Methods:

### 1. Cells and Viruses:

The myxomatosis viruses of the Toulouse 1 strain (T1), and also the modified viruses are propagated on RK13 cells in medium formed from DMEM (Dubelcco's Minimal Eagle Medium, Gibco®) supplemented with penicillin to 100 IU/ml final and streptomycin to 100 μg/ml, supplemented with 2% fetal calf serum (FCS). The selection of the recombinant viruses gpt+ was performed in the MX-HAT selection medium formed from DMEM 5% FCS supplemented with mycophenolic acid 25 μg/mL (Sigma®), with xanthine 250 μg/mL (Sigma®), with hypoxanthine 15 μg/mL, with aminopterin 0.176 μg/mL and with thymidine 4 μg/mL.

### 2. Construction of the Transfer Plasmids:

### 2.1—Transfer Plasmid for the N-gfp-M022L Fusion:

The primers used to construct this plasmid are listed in Table I below.

TABLE I

| Name | | |
|---|---|---|
| of the | | |
| primer | Sequence 5′→3′ | Use |
| F-GFP- | ggaagatctatgctatcacttttttct | Amplification |
| M022L | (SEQ ID NO: 3) | of M022L |
| R-GFP- | ttctgcagtagtaattgcactgcgt | Amplification |
| M022L | (SEQ ID NO: 4) | of M022L |
| F-pM022L | ctagctagcgtggacaatgtatcatgc | Amplification |
| | (SEQ ID NO: 5) | of the region |
| | | containing the |
| | | M022L promoter |
| R-pM022L | ctagctagcgcatttaatacatgaa | Amplification |
| | (SEQ ID NO: 6) | of the region |
| | | containing the |
| | | M022L promoter |

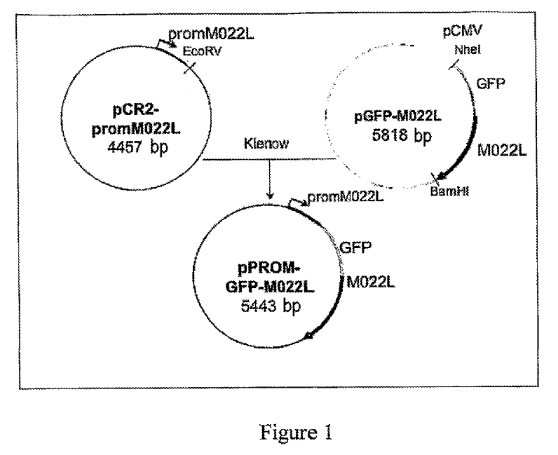

The sequence upstream of the M022L gene, containing the natural M022L promoter, was amplified with the primers F-pM022L and R-pM022L and cloned into the plasmid pCR2 (Invitrogen) resulting in the plasmid pCR2-promM022L. The M022L gene was amplified by PCR by means of the primers F-GFP-M022L and R-GFP-M022L and cloned by amino-terminal fusion with GFP (Green Fluorescent Protein) into the plasmid pEGFP-F (Clontech) resulting in the plasmid pGFPM022L. The fragment GFP-M022L was removed from pGFP-M022L by NheI and BamHI double digestion, followed by a treatment with the Klenow fragment of DNA polymerase I (Invitrogen) so as to produce blunt ends at both ends. In parallel, the plasmid pCR2-promM022L was opened with EcoRV and then dephosphorylated with shrimp alkaline phosphatase (Promega). The fragment GFP-M022L was cloned into pCR2promM022L resulting in the plasmid pPROM-GFP-M022L. These construction steps are represented schematically in FIG. 1.

2.2—Transfer Plasmid for the Fusions N-M2e-M022L and C-M2e-M071L:

The primers used are listed in Table II below.

TABLE II

| | | M071L- | M2e- |
|---|---|---|---|
| Name | Primer sequence | M2e | M022L |
| F-gpt- | CCCTCGAGAACCCACCCGCTTTTTATAG | ✓ | ✓ |
| Xho I | (SEQ ID NO: 7) | | |
| R-gpt- | CGGAATTCAGTGCCAGGCGTTGAAAA | ✓ | ✓ |
| EcoR I | (SEQ ID NO: 8) | | |
| F-M022L- | CAGCCATGGTATCACTTTTTTCTAAGCCACC | | ✓ |
| Nco I | (SEQ ID NO: 9) | | |
| F-M023R- | TACTCGAGTACCCGTTTTTCTTTCTTCTGGTTCTGG | | ✓ |
| Xho I | (SEQ ID NO: 10) | | |
| R-M022L- | CTGGGCCCGCTAAGGGAGCGTATGTGGA | | ✓ |
| Apa I | (SEQ ID NO: 11) | | |
| R-M023R- | AACTGCAGTATACACGTGCGAGGACAGG | | ✓ |
| Pst I | (SEQ ID NO: 12) | | |
| F-M070R- | CAGCCATGGTGTTGTGGTTTCAGGCATC | ✓ | |
| Nco I | (SEQ ID NO: 13) | | |
| F-M071L- | CAGCAGCATATGCGGGCGGGTACGACTTTAG | ✓ | |
| Nde I | (SEQ ID NO: 14) | | |
| R-M070R- | CGGAATTCTTGGCGGGTACGTTAATC | ✓ | |
| EcoR I | (SEQ ID NO: 15) | | |
| R-M071L- | AACTGCAGCCACGATGTACGTGATTAACGTACCCGCCAAGAA | ✓ | |
| Pst I | (SEQ ID NO: 16) | | |
| T5-prom- | TTGGAATTCTTTAATTTTAATAACTAAATGTCCCTGCTGACTG | ✓ | |
| EcoR I | AAGTTGAA [symbol] | | |
| | (SEQ ID NO: 17) | | |
| P1- | TTGGAATTCTTTAATTTTAATAAC | ✓ | |
| EcoR I | (SEQ ID NO: 18) | | |
| T6- | AGACGCCATGGATCTGTCGGAGGAGTCGTTGCACTTGCATTC | ✓ | ✓ |
| Nco I | CCAACCGT [symbol] | | |
| | (SEQ ID NO: 19) | | |
| P2- | AGACGCCATGGATCTGTCGGAGGAG | ✓ | ✓ |
| Nco I | (SEQ ID NO: 20) | | |
| T5-prom- | CGGAATTCTTAATAAATCACTCGCTCTTTTTCATGTATTAAATG | | ✓ |
| M022L | TCCCTGCTGACTGAAGTTGAA [symbol] | | |
| | (SEQ ID NO: 21) | | |
| P1-prom- | CGGAATTCTTAATAAATCACTC | | ✓ |
| M022L | (SEQ ID NO: 22) | | |

The single underling indicates the restriction sites, the wavy underlining indicates the region of mutual hybridization of the long primers.

The sequence of the selection gene gpt dependent on the early-late mixed promoter p7/5 was obtained by PCR with Taq Expand High Fidelity (Roche) on the plasmid pRB-gpt with the primers F-gpt-Xho I and R-gpt-EcoR I. The PCR product (≈750 bp) was inserted directly into the commercial vector pGED®-I (Promega) to give the vector pGEMT-gpt.

2.2.1—Construction of the Carboxy-Terminal Fusion M071L-M2e (Plasmid pGEMTM070R-gpt-M2e-M071L)

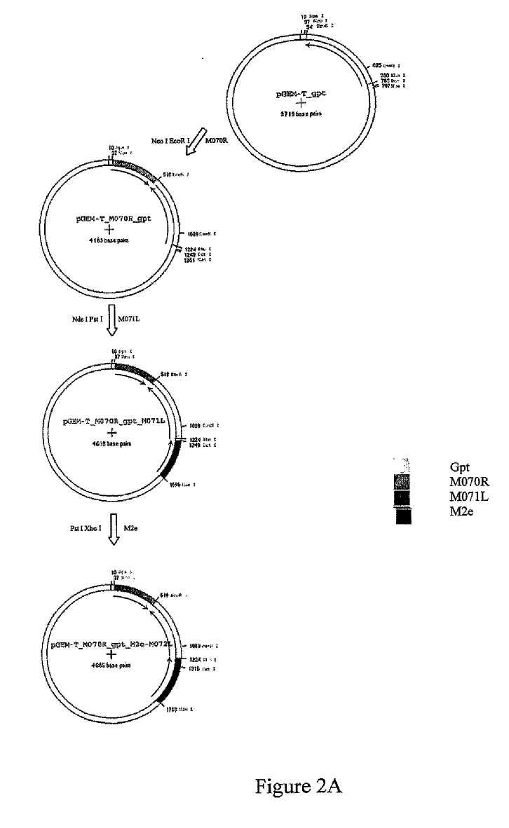

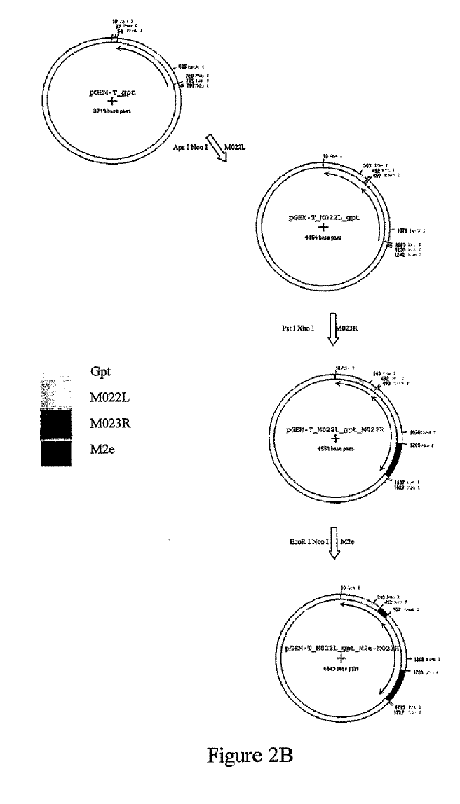

The left recombination box was obtained by PCR amplification of the 3′ sequence of the M070R gene 500 bp) of the T1 strain of myxomatosis virus with the primers F-M070RNco I and R-M070R-EcoR I. The right recombination box was obtained by PCR amplification of the 5′ sequence of the M071L gene 450 bp) of the T1 strain of myxomatosis virus with the primers F-M071L-Nde I and R-M071L-Pst I. The sequence corresponding to the ectodomain of M2 was obtained and amplified by PCR 120 bp) by means of two pairs of primers: a pair of overlapping long primers, T5-promEcoR I and T6-Nco I, and a pair of short primers corresponding to the 5′ end of each long primer, P1-EcoR I and P2-Nco I. The PCR products digested with the appropriate restriction enzymes were sequentially inserted into the vector pGEMT-gpt digested with these same enzymes. These construction steps are shown schematically in FIG. 2A. The restriction sites used are indicated to the left of the white arrows. On the right are indicated the genes or gene fragments inserted into the preceding vector by means of the same enzymes.

2.2.2—Construction of the Amino-Terminal Fusion M2e-M022L (Plasmid pGEMTM022L-gpt-M2e-M023R)

The left recombination box was obtained by PCR amplification of the 3′ sequence of the M022L gene 500 bp) of the T1 strain of myxomatosis virus with the primers F-M022LNco I and R-M022L-Apa I. The right recombination box was obtained by PCR amplification of the 5′ sequence of the M023R gene 400 bp) of the T1 strain of myxomatosis virus with the primers F-M023R-Xho I and R-M023R-Pst I. The sequence corresponding to the ectodomain of M2 was obtained and amplified by PCR 120 bp) by means of two pairs of primers: a pair of overlapping long primers, T5-prom-M022L and T6-Nco I, and a pair of short primers corresponding to the 5′ end of each long primer, P1-promM022L and P2-Nco I. The PCR products digested with the appropriate restriction enzymes were sequentially inserted into the vector pGEMT-gpt digested with the same enzymes. These construction steps are shown schematically in FIG. 2B.

### 3. Isolation of the Recombinant Viruses:

RK13 cells were infected 48 hours after culturing with T1 virus at an MOI of 0.05. After 2 hours of adsorption at 37° C., the inoculum is removed and the cells are rinsed 3 times with OptiMEM. The cells thus infected are then transfected by means of the mixture of 10 μg of plasmid DNA (each of the abovementioned transfer plasmids) and 20 μg of lipofectamine (Invitrogen) deposited on these cells for 5 hours. The cells are then rinsed 3 times and replaced in DMEM medium plus PS, 2% FCS cultured at 37° C., in 5% CO₂ until a cytopathic effect of the order of 80-90% is obtained. These recombination mixtures, formed from the cell lysate containing wild-type virus and recombinant virus undergo three freezing/thawing cycles. For the gfp-M022L fusion, ten BP100 each containing 9×10⁶ 48-hour RK13 cells are infected with the recombination mixture. After 48 hours, the liquid medium is replaced with solid medium (MEME+PS, agarose low melting point 1%, Hepes 25 mM final, NaHCO₃ 3% final, 2% FCS). After 48 hours at 37° C., under 5% CO₂, the lysis plaques containing the recombinant viruses are identified after observation by fluorescence microscopy and isolated. For the fusions M2e-M071L and M2eM022L, the RK13 cells are pretreated for 5 hours minutes with the selection medium before applying the recombination mixtures, and the inocula are then replaced with a suitable volume of selection medium, before applying the solid medium (supplemented with MPA, xanthine and HAT). Purification of the recombination viruses is then performed by several passes/isolations in limit dilution: a stock plaque of recombinant virus is isolated, subjected to three freezing/thawing cycles and plated out at different dilutions so as to isolate a daughter plaque that will again undergo this same protocol. The viruses thus obtained are then amplified and titrated.

### Results:

### Expression of the Fusion Proteins

Observation by fluorescence microscopy of RK13 cells transfected with the plasmid pGFP-M022L shows that the fusion protein is indeed expressed, and its intracellular localization appears to be similar to that described previously for the F13L protein, which is the homolog of M022L in vaccinia virus under transfection conditions (Husain et al., Virology, 308, 233-242, 2003). It thus appears that the amino-terminal fusion does not impair the production and stability of the M022L protein.

Moreover, the viruses T1-N-gfp-M022L, T1-N-M2e-M022L and T1-C-M2e-M071L form lysis plaques in RK13 cells that are comparable to those formed by the wild-type T1 virus, which indicates that these fusions do not impair the viability or viral proliferation in vitro. The observation of lysis plaques obtained after infection with the virus T1-N-gfp-M022L by fluorescence microscopy shows, moreover, that the fusion protein is expressed in all of the plaques.

### EXAMPLE 2

### Immunogenic Properties of Recombinant Myxomatosis Viruses Expressing an Exogenous Protein in Fusion with M022L or M071L

The recombinant viruses obtained in Example 1 were tested in vivo (rabbits, mice and sheep), on the one hand to explore the pathogenic power for the target species of the VM (rabbits), and on the other hand to check the establishment of an immune response against the transgene products in the target species and nontarget species of the VM.

### Materials and Methods:

### 1. Immunization and Clinical Monitoring in the Target Species:

Batches of four male New Zealand rabbits 10 weeks old were inoculated intradermally in the ear with 5×10³ pfu (plaque-forming units) of each of the recombinant viruses diluted in OptiMEM without PS or FCS. Blood samples on a dry tube were taken at D0, D10, D20 and D30 postinfection and the sera extracted. A control group of four animals was inoculated in the same manner with the same dose of T1 virus (parental virus). Clinical monitoring was performed by regular observation of the rabbits: presence of evocative lesions, evaluation of the general state.

### 3. Immunization and Clinical Monitoring in Nontarget Species:

Batches of four 8-week-old male mice of BalbC type were infected intraperitoneally with 5×10⁶ pfu of T1-N-M2e-M022L or T1-C-M2e-M071L virus at a rate of two injections with an interval of 21 days. A batch of two uninfected mice served as the control. The mice were monitored (presence of lesions, evaluation of the general state) regularly up to 42 days postinoculation, and then sacrificed and bled to extract the sera.
Two adult ewes were inoculated intradermally with 5×10⁷ pfu of virus T1-Ngfp-M022L on D0, D8 and D24, and then intramuscularly on D90. Clinical monitoring was performed by regular observation (presence of evocative lesions, evaluation of the general state) for 7 days after each inoculation, and blood samples were taken over EDTA tubes for extraction of the peripheral blood mononuclear cells (PBMC) on D0 and D110 postinoculation.

### 4. Analysis of the Sera:

The presence of anti-GFP antibodies in the sera of the rabbits inoculated with the virus T1-NgfpM022L was tested by immunofluorescence. The RK13 cells were transfected with 2 μg of plasmid pEGFP-F (Clontech) using 3 μl of Fugen6 (Roche). After culturing for 48 hours at 37° C. under 5% CO₂, the cells are fixed with 4% paraformaldehyde in PBS. Detection of the GFP by immunofluorescence is then performed. The cells are permeabilized with 0.1% Triton X100 in PBS. After rinsing, 300 μl of the sera variously diluted (1/100; 1/300) in PBS Tween are deposited on the cells. The whole is incubated for 1 hour at 37° C. Three rinses with PBS Tween are performed. Biotinylated rabbit anti-IgG secondary antibody (Sigma) diluted to 1/200 in PBS Tween is deposited in the cupules and incubated for 1 hour at 37° C. Revelation is performed by means of TRITC-coupled extravidin (Sigma): 300 μl of TRITC-coupled extravidin diluted to 1/200 in PBS are deposited in each cupule and incubated for 30 minutes at 4° C. The cells are then rinsed three times with PBS, followed by mounting on a watch glass and cover slip with PSB glycerol (50%-50%) for observation.

The presence of anti-M2e antibody in the sera of rabbits and mice inoculated with the viruses T1-N-M2e-M022L or T1-C-M2e-M071L was tested by immunofluorescence. The RK13 cells were infected with influenza virus of avian H7N1 type (MOI of 0.3) or with influenza virus of human H1N1 type (MOI of 0.1) and then fixed for 8 hours p.i. The detection of the M2 protein follows the protocol mentioned previously, the rabbit and mouse sera being diluted, respectively, to 1/200 and 1/100. The FITC-coupled mouse anti-IgG and rabbit anti-IgG secondary antibodies were used at a dilution of 1/200.

### 5. Analysis of the Cell Response in Sheep:

The cell response toward the product of the fused transgene was explored in two sheep inoculated with the virus T1-N-gfp-M022L. The principle is to measure by flow cytometry the lymphoproliferation after restimulation with gfp of PBMCs isolated and cultured in the presence of a fluorochrome (CFSE).

Isolation of the PBMCs

Blood is collected onto an EDTA tube and then diluted (1:2) with PBS. The mixture is loaded onto a density gradient (FicollPaque plus, Amersham) and then centrifuged at 800 g for 20 minutes. The PBMCs (peripheral blood mononuclear cells) are collected, washed with PBS and recovered by centrifugation at 870 g for 10 minutes at 4° C. The PBMCs are cultured at 37° C. under an atmosphere containing 5% CO₂. The RPMIc culture medium is formed from RPMI 1640 Glutamax, 25 mM Hepes (Gibco-BRL), to which is added 10% fetal calf serum (FCS), 1% of sodium pyruvate (Gibco-BRL), 1% of nonessential amino acids (Gibco-BRL), 1% of β-mercaptoethanol (Gibco-BRL), 100 units/ml of penicillin and 100 μg/ml of streptomycin.

### Lymphoproliferation Test

After isolation, the PBMCs are labeled with CFSE (Molecular Probes). The cells are taken up in PBS to a concentration of 10⁷ cells/ml. A 2X solution (2.5 μM) of CFSE in PBS is added volume-for-volume to the cells. The whole is incubated for 10 minutes in the absence of light. Next, FCS is added volume-for-volume to neutralize the labeling. The mixture is incubated for 3 minutes. The tube is then made up with RPMIc and centrifuged at 300 g for 10 minutes at 4° C. Washing is repeated in a new tube. The cell concentration is adjusted to 6×10⁶ cells/ml with RPMIc. The cells are cultured in P24 plaques at a rate of 3×10⁶ cells per well in 1 ml. Variable amounts of rGFP protein (Clontech) (10 to 0.1 μg) are added to the cells. The cells are thus cultured at 37° C. under an atmosphere containing 5% CO₂. After incubation for 6 days, the cells are harvested, washed with FACS buffer (PBS, 2.5 mM EDTA, 0.5% BSA) and labeled with A647-coupled anti-CD2 antibody and phycoerythrin-coupled anti-CD4 antibody (Sérotec). The viability is determined by adding propidium iodide at 1 μg/ml just before acquisition (BD Biosciences Pharmingen). The acquisition is performed with a FACSCalibur machine (Becton Dickinson). The analysis is performed with the Flowjo software.

### Results:

### 1—The Recombinant MVs are Nonpathogenic:

In rabbits infected with the parental virus T1, a normal evolution of the disease is observed, with death within ten to fifteen days.

In rabbits infected with the recombinant viruses, a mild inflammation located at the point of inoculation is observed. Within twenty days postinfection, all the rabbits maintained a very good general state. A few secondary myxomas of small size were occasionally observed on the eyelids, but no respiratory bacterial overinfection was observed.

As regards the nontarget species (mice and sheep), no local or general clinical signs were observed.

These data as a whole show the harmlessness of the recombinant viruses, despite the fact that they were constructed from a pathogenic strain.

2—Production of Antibodies Toward the Antigens Fused with M022L or M071L

### In a Target Species:

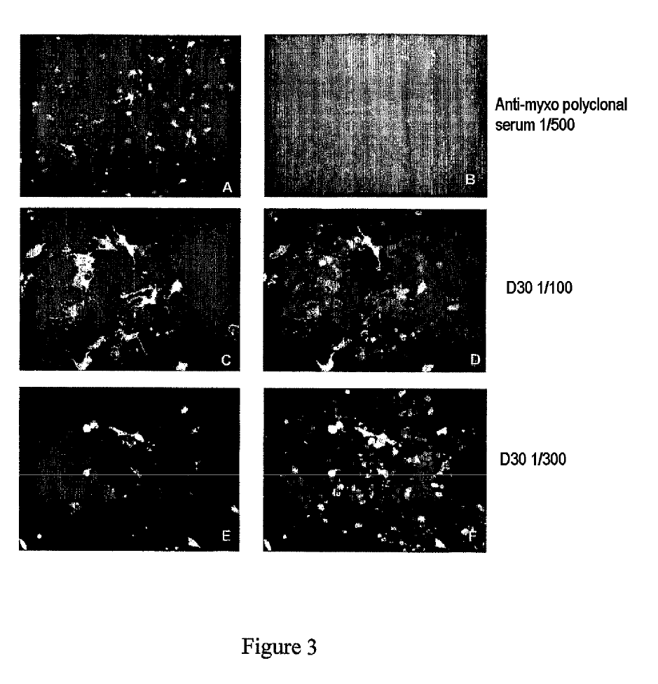

The sera of rabbits infected with the virus T1-N-gfp-M022L were collected on D0, D10, D20 and D30 postinfection. The presence of anti-GFP antibodies was tested by immunofluorescence on cells transfected with the plasmid pEGFP-F allowing transient expression of the GFP protein in eukaryotic cells. Since GFP emits in the green region, the detection was performed by means of a red fluorochrome, TRITC. The presence of anti-GFP antibodies may thus be checked by colocalization of the natural fluorescence of GFP and of the immunolabeling. The results obtained with the serum of a rabbit collected on D30 postinfection with the virus T1-N-gfp-M022L are illustrated by FIG. 3. The left-hand panels (A, C and E) show the localization of the GFP. The right-hand panels (B, D and F) show the labeling obtained using various sera: the anti-myxomatosis virus polyclonal serum (B), the rabbit serum 40451 collected on D30 postinfection diluted to 1/100 (D), and this same serum diluted to 1/300 (F).

Similarly, the presence of anti-M2e antibodies was checked for the sera of the rabbits infected with the viruses T1-N-M2e-M022L and T1-C-M2e-M071L collected on D0 and D30. To do this, RK13 cells preinfected with an influenza virus (H1N1) were subjected to an immunofluorescence test.

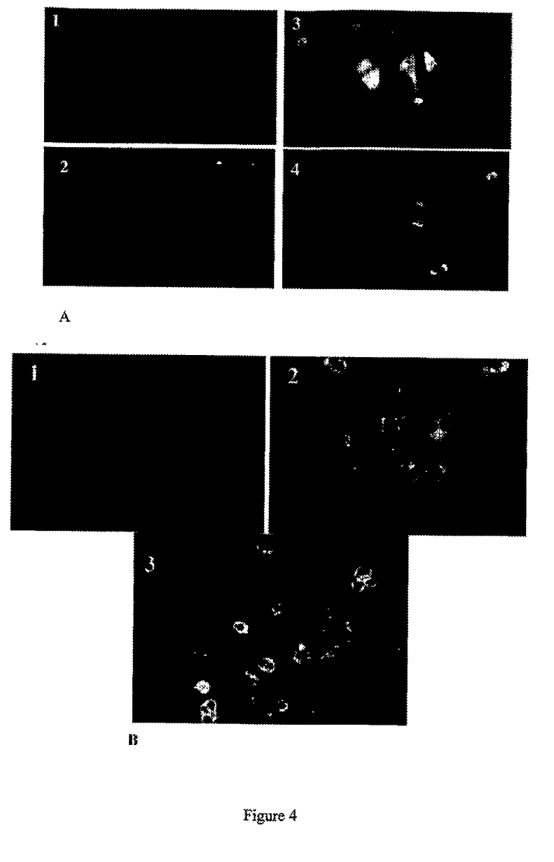

The results are illustrated by FIG. 4A: sera of rabbits infected on D0 (T1-N-M2e-M022L at 1 and T1-C-M2e-M071L at 2) and on D30 (T1-N-M2e-M022L at 3 and T1-C-M2e-M071L at 4).

The specific fluorescence observed with the sera harvested at D30 on the infected rabbits indicates the production of anti-M2e antibodies against the product of the fused transgene by the infected rabbits.

### In a Non-Target Species:

In order to check whether the recombinant viruses were capable of inducing a serological response against the transgene product in a nontarget species, mice were immunized with one or other of the viruses expressing M2e in fusion. The sera were collected 42 days after inoculation and tested by immunofluorescence.

The results are illustrated by FIG. 4B: serum of uninfected mice (1) and of mice infected with the virus T1-N-M2e-M022L (2) or the virus T1-C-M2e-M071L (3).

A specific fluorescence is observed with the sera of the mice inoculated with the viruses T1-N-M2e-M022L or T1-C-M2e-M071L, indicating the production of anti-M2e antibodies.

3. Induction of a Cell Response Against an Antigen Fused with M022L

In order to check whether a specific immune response to cell mediation can be induced against an antigen protein fused to an envelope protein of the VM, a sheep PBMC stimulation-labeling experiment was performed. The proliferation of the sheep PBMCs inoculated with the virus T1-Ngfp-M022L was measured by flow cytometry after restimulation with GFP and labeling of the subpopulations of T lymphocytes (CD2+and CD4+).

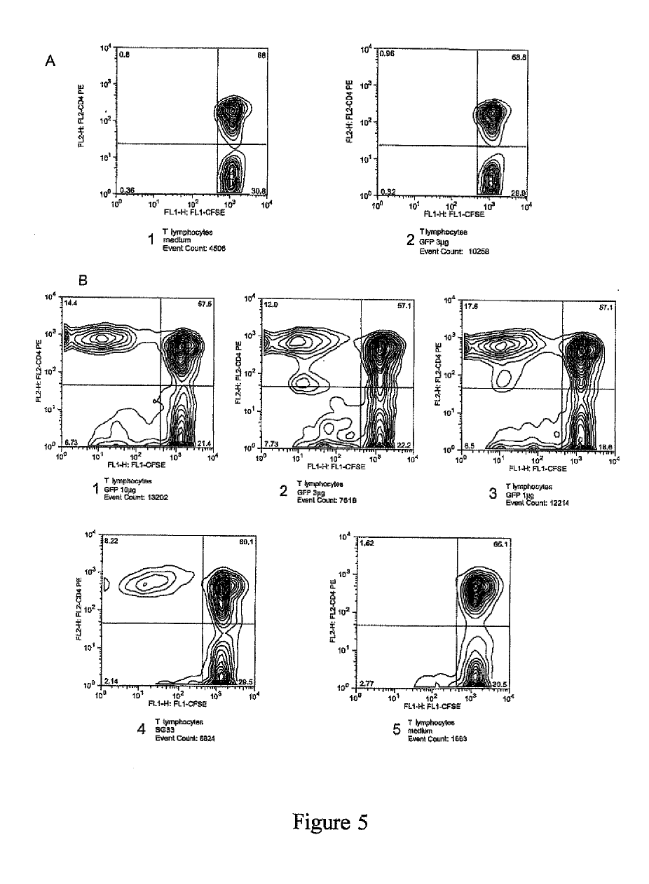

The results are illustrated by FIG. 5.

Panel A (control sheep): at 1, the cells restimulated with culture medium; at 2, cells restimulated with 3 μg of GFP.

Panel B (sheep inoculated with T1-Ngfp-M022L): at 1, 2 and 3, cells restimulated with, respectively, 10, 3 and 1 μg of GFP; at 4, cells restimulated with VM, and at 5, cells restimulated with culture medium.

For each of the graphs, the x-axis measures the intensity of the green fluorescence (CFSE) and the y-axis measures the intensity of the red fluorescence (CD4 labeling). In the corner of each graph is indicated the percentage of cells of this graph with respect to the total population CD2+ (all of the T lymphocytes).

It is observed that no dilution of fluorescence for the T lymphocyte population of the control takes place after the restimulations (maximum fluorescence intensity at 1 and 2). On the other hand, the fluorescence decreases significantly when the T lymphocytes of the inoculated sheep are restimulated with GFP, irrespective of the applied dose (panel B, 1, 2 and 3). This is more pronounced for the CDC4+ lymphocytes (upper graphs) than for the CD8+ lymphocytes (lower graphs). This decrease in fluorescence after stimulation with GFP indicates lymphoproliferation, which is evidence of a cell response in the inoculated animals.

Conclusion:

The above results illustrate the capacity of Leporipoxviruses expressing an exogenous protein antigen fused to an envelope protein of IMV or of EEV to induce both a humoral and a cellular immune response against this exogenous antigen, including in the case of species that are not Leporipoxvirus targets. In addition, the inoculation of these Leporipoxviruses does not induce any side effect, especially in the case of the nontarget species, which reinforces their harmlessness.

These Leporipoxviruses thus constituent non-replicative vaccine vectors, which are particularly advantageous for use in species other than leporidae.

1. A modified Leporipoxvirus, characterized in that it expresses a chimeric envelope protein chosen from:
a) a chimeric envelope protein comprising:
a region A formed by a protein having at least 85%, preferably at least 90% and most preferably at least 95% identity, or at least 90%, preferably at least 92% and most preferably at least 96% sequence similarity with the M071L protein defined by the sequence SEQ ID NO: 1; and
a region B formed by an exogenic protein of interest, said region B being fused to the C-terminal end of said region A;
b) a chimeric envelope protein comprising:
a region A formed by a protein having at least 85%, preferably at least 90% and most preferably at least 95% identity, or at least 90%, preferably at least 92% and most preferably at least 96% sequence similarity with the M022L protein defined by the sequence SEQ ID NO: 2; and
a region B formed by an exogenic protein of interest, said region B being fused to the N-terminal end of said region A.

2. The modified Leporipoxvirus as claimed in claim 1, characterized in that it expresses a chimeric protein as defined in a) in said claim 1, and a chimeric protein as defined in b) in said claim 1.

3. The modified Leporipoxvirus as claimed in claim 1, characterized in that it is obtained from myxomatosis virus or from rabbit fibroma virus.

4. A polynucleotide coding for a chimeric envelope protein as defined in a) or in b) in claim 1.

5. An expression cassette comprising a polynucleotide as claimed in claim 4.

6. A recombinant vector comprising an expression cassette as claimed in claim 5.

7. The modified Leporipoxvirus as claimed in claim 1, for use as a vaccine.


SEQUENCE LISTING (22 sequences)

SEQ ID NO: 1 - protein, 324 aa, Myxoma virus
MASPSKTVGVSVYVIPVTERDPFEVIPDVKEEGKRFAEVSDVKKDAKAGGINDPTKVSFYYNYYVWRKVERSGGVDNFKDYFSGLCNLLCTSESRNSMARHFSLWEEYVKTPVKDPNSAYIVVLEDDTTLLDLTTIHKTITSMVEKNIDILQLRETLHNSNARNILYQGASESMHSYTGGYDFSLSAYIIKISAATRIVEEIIRTKGISTTLSYEMYKMERALQINRQVLNEASAYVQHNTLYVSNKRVNEMKHGLWNRVSQWMSGKFPNIAYFLSHPVVSFFGLFDITILGVSIVLFIILLLVFGINSKLLWFLAGTLITYIV

SEQ ID NO: 2 - protein, 371 aa, Myxoma virus
MLSLFSKPPPGAGCRIVETVPENLGITTQHMHTHECFDELITQAKRYIHIASFCCNLRTTDQGRLIMKKLKEAAKSGVRVTILVDYQSGNKDEEELLESNVEYIKVKIGKRYNPGVLLGSFWIVDGTRCYIGNASLTGGSISNIKTLGVYSTYAPLAADLERRFSTFKAFNGNKSILSILHTACCLTTSTQYNINNPIGGVFLSDSPDYMLGCSRTLDADVVLGKISKAKKSICLELLSLVPVIREDEKTVYWPNIYNELICAAINRGVKVRLLIGSWSNNDIYVMSSVKSLQAMCSNNDLIVKVFYDKNNTKLMIVDDEFAHITPANFDGTHYLRHAFVSFNTVHPELVHMLNAIFTRDWENPRNTVVKN

SEQ ID NO: 3 - DNA, 27 bp, Artificial
  Synthetic sequence
ggaagatctatgctatcacttttttct

SEQ ID NO: 4 - DNA, 25 bp, Artificial
  Synthetic sequence
ttctgcagtagtaattgcactgcgt

SEQ ID NO: 5 - DNA, 27 bp, Artificial
  Synthetic sequence
ctagctagcgtggacaatgtatcatgc

SEQ ID NO: 6 - DNA, 25 bp, Artificial
  Synthetic sequence
ctagctagcgcatttaatacatgaa

SEQ ID NO: 7 - DNA, 28 bp, Artificial
  Synthetic sequence
ccctcgagaacccacccgctttttatag

SEQ ID NO: 8 - DNA, 26 bp, Artificial
  Synthetic sequence
cggaattcagtgccaggcgttgaaaa

SEQ ID NO: 9 - DNA, 31 bp, Artificial
  Synthetic sequence
cagccatggtatcacttttttctaagccacc

SEQ ID NO: 10 - DNA, 36 bp, Artificial
  Synthetic sequence
tactcgagtacccgtttttctttcttctggttctgg

SEQ ID NO: 11 - DNA, 28 bp, Artificial
  Synthetic sequence
ctgggcccgctaagggagcgtatgtgga

SEQ ID NO: 12 - DNA, 28 bp, Artificial
  Synthetic sequence
aactgcagtatacacgtgcgaggacagg

SEQ ID NO: 13 - DNA, 28 bp, Artificial
  Synthetic sequence
cagccatggtgttgtggtttcaggcatc

SEQ ID NO: 14 - DNA, 31 bp, Artificial
  Synthetic sequence
cagcagcatatgcgggcgggtacgactttag

SEQ ID NO: 15 - DNA, 26 bp, Artificial
  Synthetic sequence
cggaattcttggcgggtacgttaatc

SEQ ID NO: 16 - DNA, 42 bp, Artificial
  Synthetic sequence
aactgcagccacgatgtacgtgattaacgtacccgccaagaa

SEQ ID NO: 17 - DNA, 64 bp, Artificial
  Synthetic sequence
ttggaattctttaattttaataactaaatgtccctgctgactgaagttgaaactccgactcgta

SEQ ID NO: 18 - DNA, 24 bp, Artificial
  Synthetic sequence
ttggaattctttaattttaataac

SEQ ID NO: 19 - DNA, 63 bp, Artificial
  Synthetic sequence
agacgccatggatctgtcggaggagtcgttgcacttgcattcccaaccgttacgagtcggagt

SEQ ID NO: 20 - DNA, 25 bp, Artificial
  Synthetic sequence
agacgccatggatctgtcggaggag

SEQ ID NO: 21 - DNA, 81 bp, Artificial
  Synthetic sequence
cggaattcttaataaatcactcgctctttttcatgtattaaatgtccctgctgactgaagttgaaactccgactcgtaacg

SEQ ID NO: 22 - DNA, 22 bp, Artificial
  Synthetic sequence
cggaattcttaataaatcactc